(19) United States
(12) Patent Application Publication
(10) Pub. No.: US 2023/0400441 A1
(43) Pub. Date: Dec. 14, 2023

(54) IDENTIFICATION METHOD OF VOLATILE FLAVOR COMPOUND IN MEAT AND USE THEREOF

(71) Applicant: Liaocheng University, Liaocheng (CN)

(72) Inventors: Mengmeng Li, Liaocheng (CN); Changfa Wang, Liaocheng (CN); Wei Ren, Liaocheng (CN); Mengqi Sun, Liaocheng (CN); Mingxia Zhu, Liaocheng (CN); Wenqiong Chai, Liaocheng (CN); Wenqiang Liu, Liaocheng (CN); Guiqin Liu, Liaocheng (CN)

(73) Assignee: Liaocheng University, Liaocheng (CN)

(21) Appl. No.: 18/182,480

(22) Filed: Mar. 13, 2023

(30) Foreign Application Priority Data
Jun. 13, 2022 (CN) 202210663078 .6

(51) Int. Cl. G01N 30/86

(52) U.S. Cl. CPC G01N 30/8686; G01N 2030/025

(57) ABSTRACT

The present disclosure relates to an identification method of volatile flavor compounds in meat and use thereof in meat line identification. The identification method includes the following steps: step 1, sample treatment: selecting meat samples for incubation; step 2, sample analysis: analyzing gas samples of the meat samples obtained in step 1 by gas chromatography-ion mobility spectrometry (GC-IMS); and step 3, data analysis: qualitatively analyzing compounds using Library Search software of a gas chromatograph-ion mobility spectrometer to obtain a composition and peak intensities thereof, and establishing visual fingerprints according to data of relative ion peak intensities of the gas samples. According to the method, the trace volatile flavor compounds in the meat can be rapidly identified, featuring stable results, short analysis time, and visualization. The method can be used in application fields of flavor compound analysis, quality identification, origin traceability, and grade discrimination of meat products.

### CROSS REFERENCE TO RELATED APPLICATION

This patent application claims the benefit and priority of Chinese Patent Application No. 202210663078.6, filed with the China National Intellectual Property Administration on Jun. 13, 2022, the disclosure of which is incorporated by reference herein in its entirety as part of the present application.

### TECHNICAL FIELD

The present disclosure belongs to the technical field of detection and analysis of volatile substances, and particularly relates to an identification method of volatile flavor compounds in meat and use thereof.

### BACKGROUND

Meat flavor is a key factor influencing consumer choice. During meat processing, Maillard reaction and lipid degradation occur, resulting in a large number of volatile compounds that impart aroma to meat. Among them, the Maillard reaction usually produces sulfur-containing compounds, nitrogen-containing heterocyclic compounds, and oxygen-containing heterocyclic compounds. Lipid degradation usually produces aliphatic aldehydes, ketones, alcohols, acids, esters, and the like, resulting in animal-specific meat flavors. Obviously, volatile flavor compounds in meat are rich and complex in structure, and there are certain technical difficulties in identification. The identification of volatile flavor compounds is not only related to the content thereof, but also closely related to extraction and analysis methods thereof. Usually, gas chromatography-mass spectrometry or gas chromatography-olfactometry-mass spectrometry is used to identify volatile flavor compounds. Both methods require sample pretreatment, involve heating, distillation, extraction and other processes. Operations are cumbersome. The sample volume is substantially consumed, and it is easy to cause organic solvent residues. The sample pretreatment process may destroy some of the original characteristic aroma components of the meat; in addition, the pretreatment or detection time is too long, which may also lead to untimely and inaccurate determination results. Therefore, how to realize the freedom of the sample pretreatment, stable results, fast response, and high sensitivity in the detection process of volatile flavor compounds in meat is an urgent problem to be solved at present.

### SUMMARY

In order to solve the above problems, the present disclosure provides an identification method of volatile flavor compounds in meat, which can extract, rapidly separate, identify and analyze the volatile flavor substances in meat.

To achieve the above objective, the present disclosure adopts the following technical solutions:

An identification method of volatile flavor compounds in meat is provided, including the following steps:

step 1, sample treatment: selecting meat samples for incubation, where preferably, the incubation is conducted under the following conditions: incubating in a headspace bottle for 10-20 min at 50-70° C.;

step 2, sample analysis: analyzing gas samples of the meat samples obtained in step 1 by gas chromatography-ion mobility spectrometry (GC-IMS); where

gas chromatographic (GC) conditions are as follows:

a chromatographic column is MXT-5, 15 mL, 0.53 mm ID, 1 μm FT;

a column temperature is 60° C.;

a carrier gas is nitrogen (purity≥99.999%); and

a carrier gas flow is programed as follows: 0-2 min, 2 mL/min; 2-10 min, 2-20 mL/min;

and 10-20 min, 20-100 mL/min;

ion mobility spectrometry (IMS) conditions are as follows:

a drift tube temperature is 60° C.;

a drift gas is nitrogen (purity≥99.999%); and

a mobility spectrum temperature is 45° C.;

drift gas flow rate is 150 mL/min; and

positive ionization and β-ray (tritium, 3H) irradiation are used;

analysis procedure: volatile substances enter a gas chromatographic column with the carrier gas for initial separation, and enter an ion migration tube; after molecules to be tested are ionized in an ionization zone, the molecules migrate to the Faraday disk under the action of the electric field and the reverse drift gas to achieve secondary separation to obtain information on the volatile substances of the sample;

step 3, data analysis:

qualitatively analyzing compounds using Library Search software (built-in NIST and IMS databases) of a gas chromatograph-ion mobility spectrometer to obtain a composition and peak intensities thereof, and establishing visual fingerprints according to data of relative ion peak intensities of the gas samples;

directly comparing fingerprint differences (two-dimensional top views, three-dimensional spectra, and differential spectra) between samples via a Reporter plugin built in the instrument;

analyzing the fingerprints with a Gallery Plot plugin built in the instrument to visually and quantitatively compare differences in volatile substances among different samples; and

performing a dynamic principal component analysis via a Dynamic PCA plugin built in the instrument, to conduct a cluster analysis on the samples and quickly determine types of unknown samples.

The present disclosure has the following positive beneficial effects:

The method provided by the present disclosure combines the advantages of high separation capacity of GC and high sensitivity of IMS, fast response, and room temperature detection under atmospheric pressure, as well as the advantage of no need for special sample pretreatment. The method can quickly realize the identification of the trace volatile flavor compounds in meat, featuring stable results, short analysis time, and visualization. The method can be used in application fields of flavor compound analysis, quality identification, origin traceability, and grade discrimination of meat products.

### BRIEF DESCRIPTION OF THE DRAWINGS

FIG. 1 illustrates an analysis process of volatile flavor compounds in meat;

FIG. 2 is a spectrum of volatile substances in meat (top view);

FIG. 3 is a differential spectrum of volatile flavor compounds in meat;

FIG. 4 shows Gallery Plot fingerprints of volatile flavor compounds in meat; and

FIG. 5 illustrates a PCA analysis of volatile flavor compounds in meat.

### DETAILED DESCRIPTION OF THE EMBODIMENTS

The present disclosure will be described in detail below with reference to the drawings and an example, but the example of the present disclosure is not limited thereto.

An identification method of volatile flavor compounds in meat was provided, successively including the following steps:

(1) Sample treatment:

1.5 g Each of Sanfen Donkey Meat Sample and Wutou Donkey Meat Samples was Weighed, put in a 20 mL headspace bottle, and incubated for 15 min at 60° C., and 500 μL each of samples was injected.

(2) Sample Analysis:

Gas samples of the meat samples obtained in step 1 were analyzed by GC-IMS;

GC conditions were as follows:

a chromatographic column was MXT-5, 15 mL, 0.53 mm ID, 1 μm FT;

a column temperature was 60° C.;

a carrier gas was nitrogen (purity≥99.999%); and

a carrier gas flow was programed as follows: 0-2 min, 2 mL/min; 2-10 min, 2-20 mL/min;

and 10-20 min, 20-100 mL/min;

IMS conditions were as follows:

a drift tube temperature was 60° C.;

a drift gas was nitrogen (purity≥99.999%); and

a mobility spectrum temperature was 45° C.;

the drift gas flow rate was 150 mL/min; and

positive ionization and β-ray (tritium, 3H) irradiation were used.

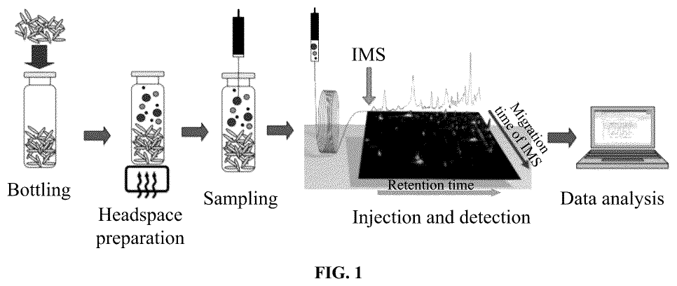

Analysis procedure: Volatile substances entered a gas chromatographic column with the carrier gas for initial separation, and entered an ion migration tube; after molecules to be tested were ionized in an ionization zone, the molecules migrated to the Faraday disk under the action of the electric field and the reverse drift gas to achieve secondary separation to obtain information on the volatile substances of the sample (FIG. 1). It can be seen that the donkey meat samples used in this application are few, and the samples do not need any special pretreatment. The GC-IMS conditions are simple and convenient to set, and the detection time is short (20 min).

(3) Data Analysis:

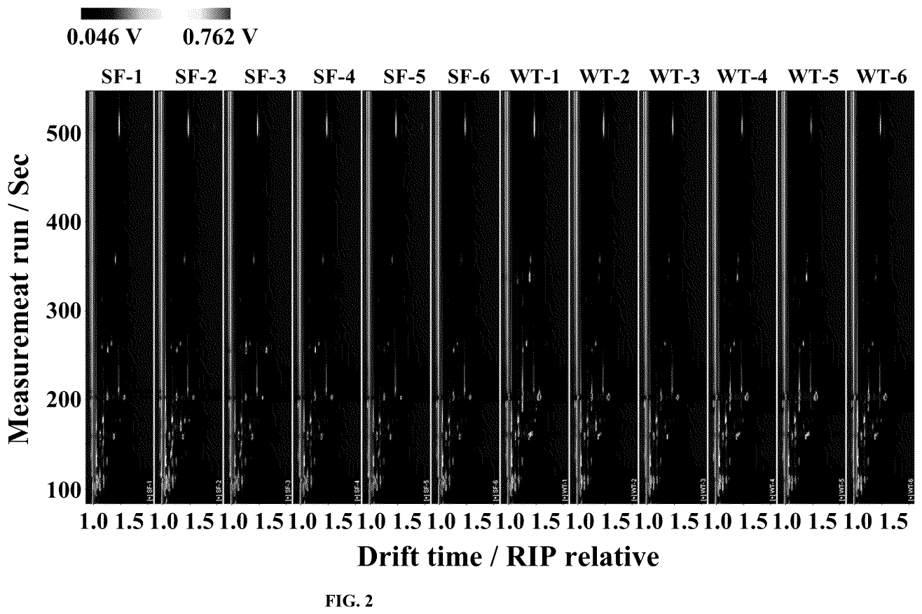

Compounds were qualitatively analyzed using Library Search software (built-in NIST and IMS databases) of a gas chromatograph-ion mobility spectrometer to obtain a composition and peak intensities thereof, and visual fingerprints were established according to the data of relative ion peak intensities of the gas samples (FIG. 2).

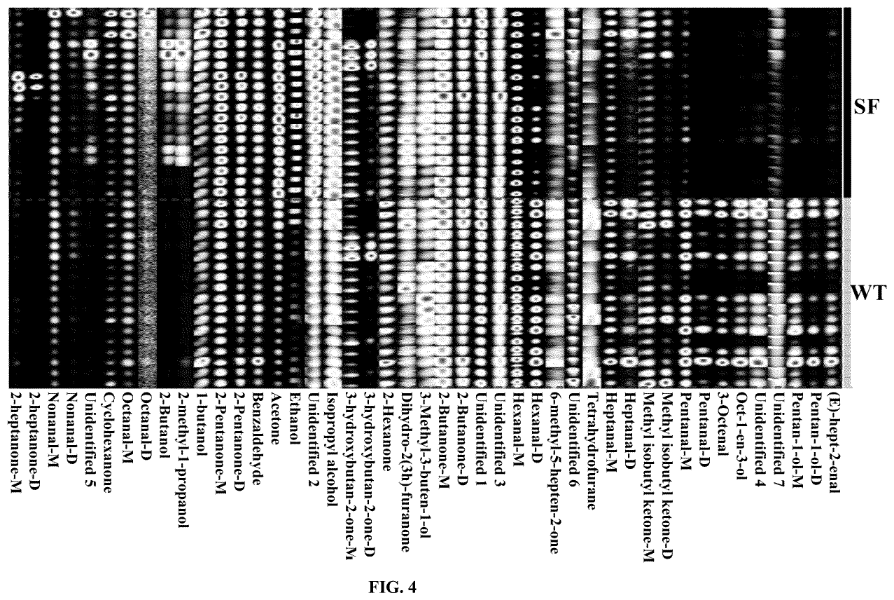

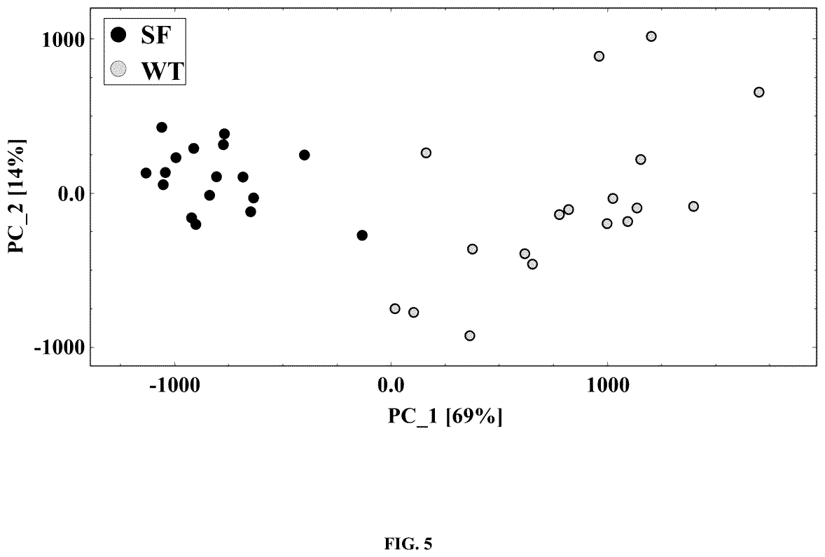

The spectral differences between donkey muscles of different lines were directly compared via the Reporter plugin; the fingerprints were analyzed using the Gallery Plot plugin (FIG. 4), which could quickly and intuitively compare the differences in volatile flavor compounds between different samples; dynamic principal component analysis (FIG. 5) was performed to conduct a cluster analysis on the samples and quickly determine types of unknown samples using the Dynamic PCA plugin.

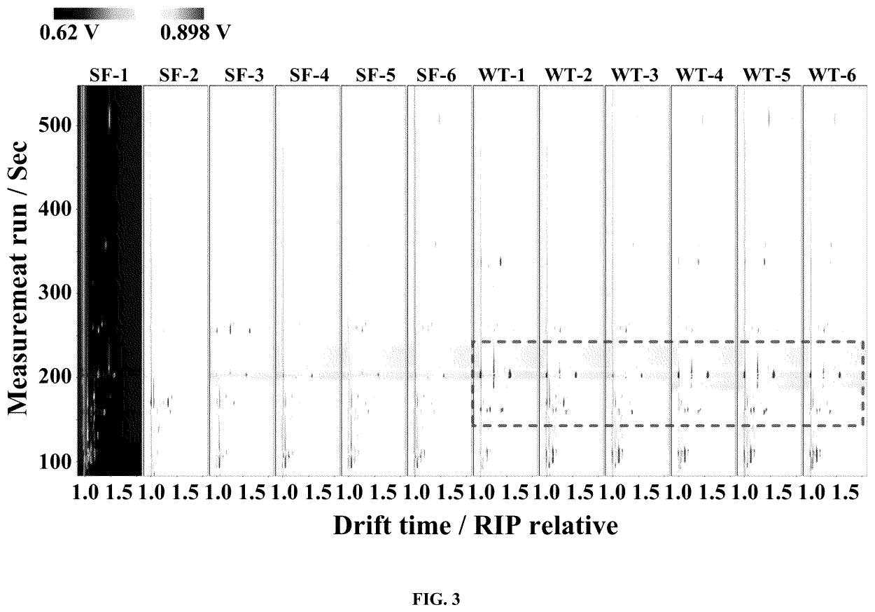

FIG. 2 is a top view of volatile flavor compounds. Combined with the differential spectrum (FIG. 3), differences in volatile flavor compounds between different samples can be visually observed. The results show the changes of relative abundance of volatile flavor compounds in the two lines of donkey meat. The fingerprints (FIG. 4) show the significantly different composition of substances, which can distinguish the characteristic volatile flavor compounds of the muscles of different Dezhou donkey breeds.

In the two lines of donkey meat, 47 volatile substances were detected and 38 were identified, including acetone, hexanal-D, hexanal-M, nonanal-M, ethanol, 3-octenal, and pentan-2-one-M. The specific flavor compounds of different lines of muscles are shown in FIG. 4, and the identification of different lines of donkey meat can be realized by different characteristic volatile flavor compounds; combined with the PCA diagram in FIG. 5, different donkey breeds can be accurately identified by the significant difference between different lines.

(4) Identification of donkey meat lines:

Six Sanfen donkey muscle samples and six Wutou donkey muscle samples were selected to carry out the operations of the above three steps in turn, respectively, and the fingerprints of each sample were obtained, which were compared and identified with the above-mentioned preset fingerprints, as shown in FIG. 3. Identification results from left to right were Sanfen donkeys (6 samples) and Wutou donkeys (6 samples), which were in full agreement with the actual results. Samples used in this detection were 1.5 g/sample, and the detection time was 20 min. The accuracy of the detection results was 100%, which could distinguish the difference among different samples, so as to identify the donkey breed. It follows that: the present application uses a few samples, the results are stable in repeatability, the detection time is short, and different lines of donkey meat can be quickly identified, with high accuracy.

The above example only describes several specific embodiments of the present disclosure, rather than limiting the scope of the patent of the present disclosure. It should be noted that persons of ordinary skill in the art can make different improvements to the drawings to obtain other drawings without creative efforts, all of which fall within the protection scope of the present disclosure. Therefore, the protection scope of the patent of the present disclosure should be subject to the appended claims.

What is claimed is:

1. An identification method of volatile flavor compounds in meat, comprising the following steps:
step 1, sample treatment: selecting meat samples for incubation;
step 2, sample analysis: analyzing gas samples of the meat samples obtained in step 1 by gas chromatography-ion mobility spectrometry (GC-IMS);
step 3, data analysis:
qualitatively analyzing compounds in the meat samples using a reference to obtain a composition and peak intensities of the compounds, and establishing visual fingerprints for the respective meat samples according to data of relative ion peak intensities of the gas samples.

2. The identification method according to claim 1, wherein establishing the fingerprint is further followed by the following analysis:
Directly comparing fingerprint differences between the meat samples.

3. The identification method according to claim 1, wherein establishing the fingerprint is further followed by the following analysis:
Analyzing the fingerprints to visually and quantitatively compare differences in volatile substances among the meat samples.

4. The identification method according to claim 1, wherein establishing the fingerprint is further followed by the following analysis:
performing a component analysis to determine types of unknown compounds in the meat samples.

5. The identification method according to claim 1, wherein the incubation is in a headspace bottle for 10-20 min at 50-70° C.

6. The identification method according to claim 1, wherein gas chromatographic (GC) conditions are as follows:
a column temperature is 60° C.;
a carrier gas is nitrogen; and
a carrier gas flow is programed as follows: 0-2 min, 2 mL/min; 2-10 min, 2-20 mL/min; and
min, 20-100 mL/min.

7. The identification method according to claim 1, wherein ion mobility spectrometry (IMS) conditions are as follows:
a drift tube temperature is 60° C.;
a drift gas is nitrogen;
a mobility spectrum temperature is 45° C.;
drift gas flow rate is 150 mL/min; and
positive ionization and β-ray irradiation are used.

8. The identification method according to claim 1, wherein a radioactive source of the β-ray is tritium, 3H.

9. The identification method according to claim 1, wherein the method is used in meat line identification.

10. The identification method according to claim 2, wherein the method is used in meat line identification.

11. The identification method according to claim 3, wherein the method is used in meat line identification.

12. The identification method according to claim 4, wherein the method is used in meat line identification.

13. The identification method according to claim 5, wherein the method is used in meat line identification.

14. The identification method according to claim 6, wherein the method is used in meat line identification.

15. The identification method according to claim 7, wherein the method is used in meat line identification.

16. The identification method according to claim 8, wherein the method is used in meat line identification.
